(19) United States
(12) Patent Application Publication
(10) Pub. No.: US 2019/0284258 A1
(43) Pub. Date: Sep. 19, 2019

(54) HAEMADIPSA SYLVESTRIS ANTITHROMBOTIC PEPTIDE SYLVESTIN AND USE THEREOF

(71) Applicants: KUNMING INSTITUTE OF ZOOLOGY, CHINESE ACADEMY OF SCIENCES, Kunming, Yunnan (CN); KUNMING LONGJIN PHARMACEUTICAL CO., LTD., Kunming, Yunnan (CN)

(72) Inventors: Ren Lai, Kunming, Yunnan (CN); Weihui Liu, Kunming, Yunnan (CN); Chengbo Long, Kunming, Yunnan (CN); Qiumin Lv, Kunming, Yunnan (CN)

(21) Appl. No.: 16/347,954

(22) PCT Filed: Nov. 8, 2017
(86) PCT No.: PCT/CN2017/109962

§ 371 Date: May 7, 2019

(30) Foreign Application Priority Data
Nov. 8, 2016 (CN) 201610979895.7

(51) Int. Cl. C07K 14/815; A61K 38/58; C12N 9/64

(52) U.S. Cl. CPC C07K 14/815; A61K 38/58; C12Y 304/21038; C12N 9/6451; C12N 9/6445

(57) ABSTRACT

Provided are a Haemadipsa sylvestris antithrombotic peptide Sylvestin and the use thereof, falling within the technical field of biomedicine. The Haemadipsa sylvestris antithrombotic peptide sylvestin can inhibit FXIIa and kallikrein, has anti-thrombus/infarction effects and alleviates injuries caused by cerebral ischemia, and can also be used in the preparation of inhibitors of FXIIa and kallikrein and drugs for anti-thrombus/infarction and anti-cerebral ischemic injuries.

The present application claims priority to Chinese Patent Application No. 201610979895.7, entitled “Haemadipsa Sylvestris Antithrombotic Peptide Sylvestin, Gene and Use thereof” filed on Nov. 8, 2016 with SIPO, and the entire content of which is hereby incorporated by reference.

### FIELD

The present disclosure provides a Haemadipsa sylvestris antithrombotic peptide Sylvestin, a variant and a use thereof, relating to the field of biomedical technology.

### BACKGROUND

Cerebrovascular disease is one of the main causes of death in middle-aged and elderly people in China, and it is also one of the research priorities of the world's health strategy. Among cerebrovascular diseases, the incidence of acute ischemic diseases ranks first with high morbidity, high mortality and high recurrence rate. The mortality due to stroke is higher than 1.5 million/year, and increases year by year with the increase of the elderly population, which seriously threatens human life health and life quality. Therefore, researches on drugs for the treatment of acute cerebral ischemia have enormous social needs and important social significance.

The main cause of acute cerebral ischemia is cerebral vascular embolism, which is due to unsmooth flow of blood caused by thrombosis in the cerebral blood vessels and thereby causes vessel infarctions. One of the key measures to treat acute cerebral ischemia is to relieve vascular embolism and restore blood supply to the ischemic area. Therefore, thrombolytic therapy has become a key link in the clinical treatment of acute cerebral ischemia. The application of thrombolytic drugs has been clinically proven to effectively relieve cerebral ischemia and becomes the main direction for the treatment of acute cerebral ischemia. Currently, only one type of drug, t-PA, has been approved by FDA, but the drug is only effective within 3 hours after attack. The target of t-PA is plasminogen, which results in a relatively high incidence of bleeding. The bleeding tendency of the drugs targeting FXIIa and kallikrein is very low, therefore, using them as targets to research and develop novel antithrombotic drugs with good efficacy and low side effects have good social and economic benefits and market prospects.

Leeches are a traditional Chinese medicine in China, which was described in the Shennong Ben Cao Jing more than 2,000 years ago. The Compendium of Materia Medica recorded: “Leeches can eliminate blood amassment, which is a liver meridian blood aspect drug, therefore it can unblock the liver meridian and gather the blood.” The traditional Chinese medicine believes that leeches is a traditional blood-breaking drug, have effects of expel stasis, unblocking meridians and smoothing waterway, and it is mainly used to treat diseases such like blood stasis and amenorrhea, wind stroke and hemi-paresis, traumatic injury. Europe officially approved leech therapy as a legal treatment in 2005: There are 350,000 leeches for medical care every year even in Germany alone.

Haemadipsa sylvestris, also known as Whitmania pigra Whitman, Clitellata, Haemadipsidae. The body is slightly elliptical and is about 3 cm long. The typical habitat of the species is Karen Hills, Myanmar. The distribution of Haemadipsa sylvestris is in Indonesia, Myanmar, India, Vietnam, Yunnan of mainland China, etc. The main inhabits of Haemadipsa sylvestris are wet mountain grasslands or bamboo forests, near water or in water. When human or animals pass, they attach and ingest blood. Haemadipsa sylvestris can secrete anticoagulant substances and destroy blood clotting function. Therefore, bleeding from wounds bitten by Haemadipsa sylvestris is unlikely to stop. So in folk, this feature is also adopted to use Haemadipsa sylvestris to treat localized poor blood flow of patients.

For the first time, the inventors found an FXIIa and kallikrein inhibitor from Haemadipsa sylvestris, which has strong effects on anti-thrombus/anti-acute cerebral ischemia and is named Sylvestin (hereinafter referred to as Haemadipsa sylvestris antithrombotic peptide).

The Haemadipsa sylvestris antithrombotic peptide Sylvestin according to the present disclosure can inhibit thrombosis. It has been confirmed by animal models that it has very strong anti-thrombus and acute cerebral ischemia inhibition activities. The inventors searched and compared the full amino acid sequence of the antithrombotic peptide Sylvestin with sequences in protein databases and did not find any identical polypeptide. The inventors also searched and compared the coding sequence of the Haemadipsa sylvestris antithrombotic peptide Sylvestin with gene databases and did not find any identical gene.

### SUMMARY

The object of the present disclosure is to provide a novel polypeptide Sylvestin and variants thereof having strong antithrombotic properties, as well as its prophylactic and therapeutic use, based on the inventors' above-mentioned unexpected findings.

In order to achieve the object of the present disclosure, the present disclosure provides the following technical solutions.

The molecular weight of the antithrombotic peptide Sylvestin is 4790.5 Daltons, and its amino acid sequence (SEQ ID NO: 1) is: TSEPVCACPK MLFWVCGKDG ETYTHPCIAK CHNVEVEHDG KCK.

Cloning of the cDNA corresponding to the antithrombotic peptide comprises steps of:

Haemadipsa sylvestris total RNA extraction, mRNA purification, mRNA reverse transcription and cDNA library construction, primer design, screening the gene of the Haemadipsa sylvestris antithrombotic peptide using PCR. The amplification primer is 20 nucleotides in length and the sequence is: 5′AAACCTCGGAACCGGTATGT 3′; the other amplification primer for PCR is a 3′ primer and the sequence is: 5′CCGAGGTTTGGTGGCTCATT 3′. The obtained positive single clones are subjected to nucleotide sequencing and the result shows that the DNA encoding the Haemadipsa sylvestris antithrombotic peptide is consisted of 316 nucleotides, and the sequence from the 5′ end to the 3′ end (SEQ ID NO: 2) is:

GGGAACCCGAAACGGGCATTCGAGCTCGGTACCCGGGGATCCTCTA
GAGATTAAACCTCGGAACCGGTATGTGCATGCCCAAAAATGCTATT
TTGGGTTTGTGGTAAAGATGGTGAGACTTACACCCATCCTTGCATT
GCAAAATGCCATAATGTTGAAGTTGAACATGATGGGAAGTGCAAAT
GAAAGGGACCATTCTTCGAAATTGCCTGAAACTTAAAAATATTGAT
TTGAATTTAATTAATTCTTATTAATTATAACGTTTCATCATAATAA
ATGAATTACGAAAAAAAAAAAAAAAAAAAAAAAAAAAAAA

Wherein the nucleotide fragment of 55-183 encodes the mature peptide of the Haemadipsa sylvestris antithrombotic peptide.

Isolation and purification method of the Haemadipsa sylvestris antithrombotic peptide:

passing the collecting homogenate of Haemadipsa sylvestris sample through a Sephadex G-50 chromatography column, collecting the fraction with anti-thrombus activity, lyophilizing, passing through reversed phase high-performance liquid chromatography (RP-HPLC) C4 and C18 columns, purifying to obtain a Haemadipsa sylvestris antithrombotic peptide.

Chemical synthesis method of the Haemadipsa sylvestris antithrombotic peptide:

synthesizing the full sequence using an automatic peptide synthesizer (433A, Applied Biosystems) according to the sequence determined by the Edman degradation and the amino acid sequence predicted by the gene encoding the Haemadipsa sylvestris antithrombotic peptide; desalting and purifying by HPLC reverse phase column chromatography to make sure its purity is greater than 95%; determining the molecular weight by Matrix Assisted Laser Desorption/Ionization time-of-flight mass spectrometry (MALDI-TOF). The synthesized antithrombotic peptide is dissolved in sterile water for activity assays.

The beneficial effects of the present invention are: obtain Haemadipsa sylvestris antithrombotic peptide Sylvestin by isolation and purification, obtain cDNA sequence thereof by cloning; the antithrombotic peptide can inhibit the function of FXIIa and kallikrein, showing extremely significant anti-thrombus and inhibiting acute cerebral ischemia effects; in addition, the antithrombotic peptide has the advantages of simple structure, easy synthesis and strong anti-thrombus activity, which can be used to prepare agents for inhibiting FXIIa and kallikrein and drugs for anti-thrombus and treatment of acute cerebral ischemia.

### BRIEF DESCRIPTION OF DRAWINGS

FIG. 1 shows the anti-thrombus effect of Haemadipsa sylvestris antithrombotic peptide Sylvestin.

FIG. 2 shows the anti-acute cerebral ischemic effect of Haemadipsa sylvestris antithrombotic peptide Sylvestin.

### DETAILED DESCRIPTION

The substantial content of the present disclosure will be further illustrated by the following embodiments, but the scope of the present disclosure is not limited thereto.

### Example 1 Isolation and Purification of the Haemadipsa sylvestris Antithrombotic Peptide Sylvestin

### 1.1 Preparation of Haemadipsa sylvestris Sample

Living Haemadipsa sylvestris was quickly frozen by liquid nitrogen directly. At a low temperature, the haemadipsa was wrapped with cloth and broken into pieces with a hammer. The haemadipsa pieces and an appropriate amount of 50 mM Tris-HCl (pH 8.0) buffer were mixed and homogenized. The mixture was centrifuged at 12000 g at 4° C. for 30 minutes, and the supernatant was a crude sample of the Haemadipsa sylvestris body. All the supernatants were mixed together, divided into aliquots (3 ml/tube) and stored at −80° C.

### 1.2 Sephadex G-50 Gel Isolation and Reversed Phase High-Pressure Chromatography Isolation

Step 1. Sephadex G-50 gel chromatography. 2 mL of the haemadipsa homogenate was loaded to a Sephadex G-50 (Amersham Bioscience) gel column (26 mm×100 cm) equilibrated with a Tris-HCl (0.02 mol/l, pH 7.8) buffer. Elution was carried out with the same concentration of an equilibration buffer at a flow rate of 0.3 ml/min, 3 ml/tube, and fractions were collected using a CBS-A program-controlled automatic fraction collector (Shanghai Qingpu Huxi Instrument Factory). The O.D. values at 280 nm and 215 nm were measured using an Ultrospec 2100 pro spectrophotometer (Amersham Biosciences). Each peak fraction was collected and stored at −20° C. for use.

Step 2. C4 reversed phase high-pressure chromatography. The fraction having activity was continued to be separated using a reversed phase high-pressure chromatography (Waters 1525 Binary HPLC Pump) C4 column (Lichrospher 10×250 mm); solvent A: 0.1% TFA in ultrapure water solution, solvent B: 0.1% TFA in acetonitrile; linear concentration gradient for elution: 0-10 min, B: 0%; 10-11 min, B: 0-5%; 11-40 min, B: 5-33%; 40-50 min, B: 33-38%; 50-60 min, B: 38-70%; 60-70 min, B: 70-100%; flow rate: 1.5 ml/min; loading sample: 3 mg of crude protein. Peaks were detected using a Waters 2489 Visible/UV detector (215 nm), and each peak was collected as a unit.

Step 3. C8 reversed phase high-pressure chromatography. C8 column (X Bridge™ 4.6×250 mm) was used. Solvent A: 0.1% TFA in ultrapure water solution, solvent B: 0.1% TFA in acetonitrile; linear concentration gradient for elution: 0-10 min, B: 0%; 10-14 min, B: 0-20%; 14-24 min, B: 20%; 24-55 min, B: 20-35%; 55-60 min, B: 35-100%; flow rate: 0.7 ml/min; loading sample: 1 mg of crude protein. Peaks were detected using a Waters 2489 Visible/UV detector (215 nm), and each peak was collected as a unit. The fraction having FXIIa and kallikrein inhibition activity was detected using the above steps. The purified Haemadipsa sylvestris antithrombotic peptide obtained by the purification was subjected to N-terminal sequencing using Edman degradation (model 491, ABI, USA). The molecular weight of the antithrombotic peptide was determined by Electrospray Ionisation Mass Spectrometry (ESI-MS).

### Example 2 Haemadipsa Sylvestris Antithrombotic Peptide cDNA Cloning

### 2.1 Total RNA Extraction from Haemadipsa sylvestris

(1) Several haemadipsa were removed from liquid nitrogen quickly, and the front end (including the mouth and throat) of the haemadipsa was cut with scissors; about 100 mg (total weight) sample was put into a mortar, ground thoroughly after liquid nitrogen was added. After the sample became powders, 1 mL of Trizol extraction buffer (Invitrogen) was added and ground together with liquid nitrogen. After the Trizol melted, all the liquid in the mortar was transferred to a 1.5 mL centrifuge tube and allowed to stand at room temperature for 5 min.

(2) 200 μL of chloroform was added to the tube, vigorously vortexed and mixed for 15 s, allowed to stand at room temperature for 5 min; the tube was centrifuged at 12000 g at 4° C. for 10 min; the upper colorless aqueous phase was pipetted to a new 1.5 mL centrifuge tube (pipetted as much upper phase liquid as possible, but avoided the middle or lower phase).

(3) An equal volume of isopropanol (pre-cooled at 4° C.) was added to the tube, inversed and mixed, allowed to stand at room temperature for 15 min; the tube was centrifuged at 12000 g at 4° C. for 10 min; the small amount of precipitation at the bottom of the tube was RNA, and the supernatant was removed with a pipette.

(4) 1 mL of 75% ethanol (pre-cooled on ice) was added to the precipitation for washing; the tube was centrifuged at 7500 g at 4° C. for 5 min and the supernatant was discarded. The washing was repeated twice. The tube was placed in a laminar flow cabinet for 5 to 10 min to dry (until no ethanol) to obtain the Haemadipsa sylvestris total RNA. 30 μL of 0.1% DEPC water was added to the tube after drying, gently vortexed to dissolve the RNA. 2 μL of RNA sample was added to 98 μL of DEPC water, and the absorbance was measured at 230 nm, 260 nm, and 280 nm wavelength. 10 μL of RNA sample was subjected to 1% agarose gel electrophoresis. The other total RNA was frozen and stored at −80° C.

### 2.2 cDNA Library Construction

Haemadipsa sylvestris cDNA library was constructed according to the instruction of Creator™ SMART™ cDNA Library Construction Kit (Clontech). The specific operations are as follows:

### (1) First-Strand cDNA Synthesis (mRNA Reverse Transcription)

2 μL of Haemadipsa sylvestris total RNA, 1 μL of SMART™ IV oligonucleotide and 1 μL of CDS III/3′ Primer were added to a RNase-free PCR tube, 1 μL of DEPC water was added to bring total volume up to 5 μl, mixed well and centrifuged for 10 s; the tube was heated at 72° C. for 2 min, and then incubated on ice for 2 min; 2 μL of 5×first strand buffer, 1 μL of 20 mM DTT, 1 μL of 10 mM dNTP Mix and 1 μL of PowerScript reverse transcriptase were added to the PCR tube, mixed well and centrifuged for 10 s. The tube was insulated at 42° C. for 1 h in a PCR machine, and then put on ice to terminate the reaction.

### (2) Amplification of the Second-Strand cDNA Using Long-Distance PCR (LD-PCR)

1 μL of first-strand cDNA, 40 μL of deionized H₂O, 5 μL of 10×buffer, 1 μL of 50×dNTP Mix, 1 μL of 5′ PCR primer, 1 μL of CDS III/3′ PCR primer and 1 μL of polymerase were mixed well in a PCR tube. PCR amplification was performed as follows:

i) 95° C. 1 min

ii) 20 cycles of:
95° C. 15 sec,
65° C. 30 sec,
68° C. 6 min.

After the amplification, the synthesized double-strand cDNA were put into PCR tubes (10 μL/tube). 5 μL sample was subjected to 1% agarose electrophoresis, the other was immediately stored at −80° C.

### 2.3 Preparation of E. coli DH5α Competent Cells

(1) A single DH5α colony was picked and inoculated in 1 mL of LB liquid medium without ampicillin, cultured at 37° C. overnight. The next day, the bacterial solution was inoculated again in 1 mL of LB medium at a ratio of 1:100, vortexed at 37° C. for 2 h.

(2) When the OD 600 of the culture reached 0.35, the bacterial solution was allowed to stand on ice for 10 min to be cooled to 0° C.

(3) The cells were collected by centrifuging at 5000 rpm at 4° C. for 5 min.

(4) The medium was discarded, and the tube was inverted for 1 min to let the last trace of medium run out.

(5) For every 1 mL of initial culture, 600 μL of pre-cooled 0.1 M CaCl₂-MgCl₂ solution (80 mM MgCl₂, 20 mM CaCl₂) was added to resuspend cell pellet.

(6) The cells were collected by centrifuging at 5000 rpm at 4° C. for 5 min.

(7) The medium was discarded, and the tube was inverted for 1 min to let the last trace of medium run out.

(8) For every 1 mL of initial culture, 60 μL of 0.1 M CaCl₂ pre-cooled by ice was added to resuspend cell pellet, and then the tube was put in a refrigerator at 4° C. for 10 to 18 h.

### 2.4 cDNA Library Screening

### (1) Synthesis of Specific Primers

Two primers were designed using primer blast and synthesized by Sangon Biotech. The amplification primer was 20 nucleotides in length, and the sequence was 5′AAACCTCGGAACCGGTATGT 3′; the other amplification primer was a 3′ primer, and the sequence was 5′CCGAGGTTTGGTGGCTCATT 3′.

### (2) Cloning Target Sequence from Haemadipsa cDNA Library

20 μL system: 0.1 μL of Taq enzyme, 0.4 μL of CDSIII and SMART4 respectively, 0.4 μL of dNTP, 2 μL of buffer, 1.2 μL of Mg²⁺, 16 μL of PCR water.

### PCR Condition

| | | |
|---|---|---|
| 1. Pre-denaturation | 95° C., 5 min | |
| 2. Denaturation | 95° C., 30 s | 33 Cycles |
| 3. Annealing | 56° C., 30 s | |
| 4. Extension | 72° C., 30 s | |
| 5. Final Extension | 72° C., 10 min | |

Ligation, Transformation and Detection. 0.2 μL of Takara PMD19-T vector and 2.3 μL of double-strand DNA were added to the a microcentrifuge tube; 2.5 μL of the same amount of ligase buffer mixture (dissolved on ice) was added; the tube was incubated at 16° C. overnight; all the 5 μL of the ligation product was added to 60 μL DH5α competent cells, put on ice for 30 min; the competent cells were subjected to heat shock for 90 s, gently put on ice for 3 to 5 min to let the cell membrane to be repaired; pre-warmed LB medium was added as soon as possible, and the tube was incubated in a 80 rpm shaker at 37° C. for 45 min; 100 μL cell was put onto a LB plate containing 100 μg/mL ampicillin, incubated at 37° C. for 16 h; after the colonies appeared, single clones were picked and subjected to PCR (10 μL reaction system).

### 2.5 Sequencing and Sequence Screening

Twenty single colonies, which have similar fragment size with the target sequence in PCR, were picked and subjected to DNA sequencing. An ABI 3730 sequencer was used for the sequencing. The sequencing primer was M13 (+): 5′-CGCCAGGGTTTTCCCAGTCACGAC-3′, M13 (−): 5′-GAGCGGATAACAATTTCACACAGG-3′. The obtained sequences were subjected to sequence screening.

### 2.6 Haemadipsa sylvestris Antithrombotic Peptide Sylvestin DNA Sequence Determination

Plasmid DNA was extracted and the nucleotide sequence was determined by the dideoxy method, the instrument used was a U.S. Applied Biosystems 373A automatic nucleotide sequencer. The sequencing primer was BcaBESTTM Sequencing Primer RV-M and BcaBESTTM Sequencing Primer M13-47. The sequence of BcaBESTTM Sequencing Primer RV-M was: 5′ GAGCGGATAACAATTTCACACAGG 3′, and the BcaBESTTM Sequencing Primer M13-47 was: 5′ CGCCAGGGTTTTCCCAGTCACGAC 3′. The cDNA sequencing result from the 5′ end to the 3′ end (SEQ ID NO: 2) was:

GGGAACCCGAAACGGGCATTCGAGCTCGGTACCCGGGGATCCTCTA
GAGATTAAACCTCGGAACCGGTATGTGCATGCCCAAAAATGCTATT
TTGGGTTTGTGGTAAAGATGGTGAGACTTACACCCATCCTTGCATT
GCAAAATGCCATAATGTTGAAGTTGAACATGATGGGAAGTGCAAAT
GAAAGGGACCATTCTTCGAAATTGCCTGAAACTTAAAAATATTGAT
TTGAATTTAATTAATTCTTATTAATTATAACGTTTCATCATAATAA
ATGAATTACGAAAAAAAAAAAAAAAAAAAAAAAAAAAAAA

As shown above, the nucleotide sequence of the cDNA encoding Haemadipsa sylvestris antithrombotic peptide Sylvestin cloned in this example has the following characteristics: the sequence length, 316 bases; sequence type: nucleic acid; number of strand: single strand; topology: linear; sequence type: cDNA; source: Haemadipsa sylvestris.

Analyzing with reference to Edman degradation N-terminal sequencing and mass spectrometry identification, the coding region of the Haemadipsa sylvestris mature antithrombotic peptide Sylvestin is nucleotides 55-183 of the nucleotide sequence SEQ ID NO: 2. The amino acid sequence (SEQ ID NO: 1) of the Haemadipsa sylvestris antithrombotic peptide Sylvestin is:

TSEPVCACPK MLFWVCGKDG ETYTHPCIAK CHNVEVEHDG
KCK.

The present disclosure also provided a use of the polynucleotide encoding the antithrombotic peptide Sylvestin as an antithrombotic peptide prepared in genetic engineering.

The present disclosure also provided a use of the antithrombotic peptide Sylvestin in the preparation of FXIIa and kallikrein inhibitors and anti-thrombus, anti-acute cerebral ischemia drugs.

### Example 3 Chemical Synthesis of Haemadipsa sylvestris Antithrombotic Peptide Sylvestin

3.1 Chemical synthesis of the antithrombotic peptide Sylvestin: referring to Example 2, the amino acid sequence was deduced based on the cDNA sequence and the sequencing result of the protein encoding Haemadipsa sylvestris antithrombotic peptide Sylvesti, and the full sequence was synthesized by an automatic peptide synthesizer, desalted and purified by HPLC reverse phase C18 column chromatography.

Fast atom bombardment mass spectrometry (FAB-MS) was used to determine the molecular weight, Glycerin:3-nitrobenzyl alcohol:dimethyl sulfoxide (1:1:1, V:V:V, volume ratio) were used as substrates, Cs⁺ was used as a bombardment particle, the current was 1 μA and the emission voltage was 25 Kv.

3.3 The purity of the purified Haemadipsa sylvestris antithrombotic peptide was determined by HPLC, the molecular weight was determined by fast atom bombardment mass spectrometry, the isoelectric point was determined by isoelectric focusing electrophoresis, and the amino acid sequence was determined by automatic amino acid sequencer.

Haemadipsa sylvestris antithrombotic peptide Sylvestin is a polypeptide encoded by the Haemadipsa sylvestris antithrombotic peptide gene, which has a molecular weight of 4790.5 Daltons and an isoelectric point of 6.28. The amino acid sequence of the antithrombotic peptide Sylvestin is: TSEPVCACPK MLFWVCGKDG ETYTHPCIAK CHNVEVEHDG KCK (SEQ ID NO:1).

### Example 4 Pharmacological Experiment of Haemadipsa sylvestris Antithrombotic Peptide Sylvestin

### 4.1 Enzyme Kinetics

In a 96-well plate, 10 μL of the sample and 10 μL of FXIIa with a final concentration of 0.2 μM were mixed in 40 μL of buffer (100 mM NaCl, 50 mM Tris-HCl (pH 8.0), 5 mM CaCl₂), allowed to stand at room temperature for 5 min. A mixture of 30 μL of buffer and 10 μL of chromogenic substrate with a final concentration of 0.04 mM was added to each well, the final volume was 100 μL. The kinetics of the coagulation reaction was measured using an Epoch (BioTek) microplate reader and GEN CHS 1.09 software, OD 405 nm, 20 min, with 47 s intervals. The concentration of the sample was 10 μM. The result showed that the Sylvestin synthesized in Example 3 had no inhibitory effect on thrombin, plasminogen FXa or the like, while it can inhibit FXIIa and kallikrein. The inhibition constants of Sylvestin on FXIIa and kallikrein were calculated to be 2.9 μM and 17.8 nM, respectively.

### 4.2 APTT and PT Experiments

The APTT reagent was equilibrated to room temperature and the APTT reagent was mixed by gentle inversion; 50 μL of the reagent, 50 μL of normal plasma and 5 μL of the sample were mixed and incubated in a 37° C. water bath for 3 min; 50 μL of pre-warmed CaCl₂ solution was added, mixed immediately; the OD 650 nm was detected with a microplate reader. PT experiment: prothrombin reagent was pre-warmed at 37° C. for 15 min; 50 μL of normal plasma and 5 μL of the sample were incubated in a 37° C. water bath for 3 min; 100 μL of pre-warmed prothrombin reagent was added, mixed immediately; OD 650 nm was detected with a microplate reader. The result showed that the Sylvestin synthesized in Example 3 had no effect in PT experiment, while had a concentration-dependent effect in APTT experiment, indicating that Sylvestin inhibited the endogenous coagulation pathway, consistent with the result of FXIIa inhibitor.

### 4.3 Carrageenan-Induced Rat Tail Thrombosis Model

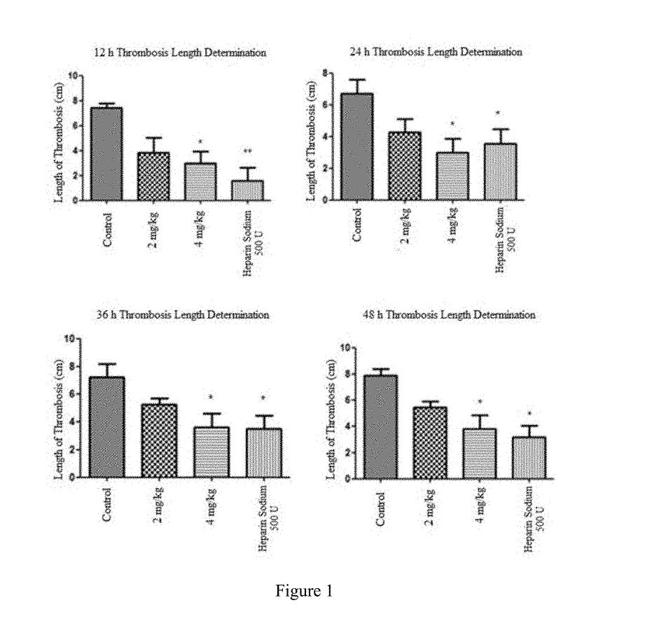

Kunming mice were used as experimental animals, body weight 25 to 30 g (provided by the Experimental Animal Center of Kunming Medical College). After one week of housing, they were randomly divided into groups (n=6), half male and half female. One group was a physiological saline control group, the sample groups were Sylvestin synthesized in Example 2 at a dose of 2 mg/kg and 4 mg/kg, respective, and the positive control was given heparin sodium (Beijing Dingguo Changsheng Biotechnology Co., Ltd.), 500 U/mouse. 30 minutes after tail vein administration, carrageenan (carrageenan, type I, Sigma, dissolved in physiological saline to a concentration of 1%) was injected from the abdomen of the mice at a dose of 60 mg/kg, since the thrombosis rate was >90% in a low temperature environment, the housing temperature was set at 17.5° C. After 12, 24, 36, and 48 h, the average length of thrombus was determined according to the color change of the tail skin. Referring to FIG. 1, the average length of thrombus in each group increased with time. At 12 h, since thrombosis was not obvious, error in the measurement was relatively high, but the symptom of the control group was more obvious than the other group's; in heparin sodium group, the length of thrombus doubled from 12 h to 24 h; the control group was basically unchanged, possibly due to the length of the rat tail; the length of the sample group increased slightly. At each measurement time point, the effects of Sylvestin and heparin on rat tail thrombosis were statistically significant compared with the control group. Data processing was Graphpad Prime 5, data mean±SD, t-test (*p<0.05). Overall, the inhabitation rate of Sylvestin against thrombosis decreased over time, but even after 48 h, the Sylvestin-treated group still showed significant inhibitory effect compared with the control group. Compared with the heparin group and the 2 mg/kg group, the inhibition rate of the 4 mg/kg group did not change much. Moreover, the inhibition rate of Sylvestin against thrombus increased as concentration increased (FIG. 1).

### 4.4 Acute Cerebral Ischemic Intraluminal Thread Model

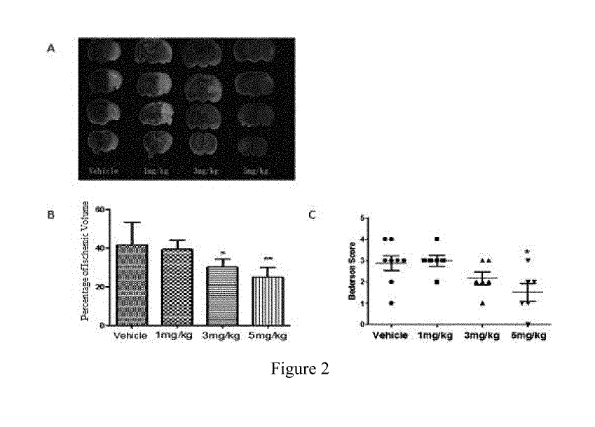

Kunming mice (30 to 35 g) were anesthetized with 2% sodium pentobarbital (80 mg/kg). After medium-deep anesthesia, a midline neck incision was made, the skin and subcutaneous tissue of the mice were incised layer by layer, the sternocleidomastoid muscle was separated, the anterior belly of the digastric muscle was incised off, the right common carotid artery (CCA), internal carotid artery (ICA) and external carotid artery (ECA) were exposed, an electric coagulator was used to coagulate thyroid artery and pharyngeal artery over the ECA and they were cut off. Ligation of the distal end of the ECA was performed, seton was placed at the proximal end, CCA and ICA were temporarily closed using a clip, the ECA was cut off, an intraluminal thread was inserted into the ICA from the ECA stump, ligation of the ECA stump was performed, the artery clip of the ICA was removed, pushed inward and upward from the ICA. Direction was appropriately adjusted, inserted to the intraluminal thread mark (10 mm from the CCA junction), timing started, the intraluminal thread was removed after 1 h, after no active bleeding was observed, incision was stitched. The temperature was maintained during the whole process using a heating blanket, the temperature was 36 to 37° C. Awakened animals were put back to the cage and were allowed to eat and drink freely. Cerebral ischemia for 24 h, and the neurological deficit score was assessed and recorded according to the Bederson Scale assessment: 0 is no dysfunction; 1 is being unable to extend the right forelimb; 2 is rotating to right; 3 is dumping to right; 4 is no autonomous activity with disturbance of consciousness; 5 is death. Anesthetized after 24 h and head was cut down, brain tissue was removed, placed in a brain mold (on ice), cut into 2 mm thick sections along the optic chiasm, and placed in a 2% TTC phosphate buffer, dyed in a 37° C. incubator for 30 min, fixed with 4% paraformaldehyde overnight and images were acquired. Percentage of ischemic volume=[ischemic volume−(volume of the left hemisphere of the brain−volume of the right hemisphere of the brain]/volume of the right hemisphere of the brain×100. 10 min before ischemia reperfusion, intravenously administrated once through tail vein, the experimental group was administrated 1, 3, 5 mg/kg of Sylvestin (synthesized according to Example 2), and the control group was administrated physiological saline, the volume was 100 μL. Within 10 min after the ischemia reperfusion, intravenously administrated once through tail vein, the dose was the same as above; administrated again after 6 h. 6 in each group. The result showed that Sylvestin can effectively alleviate the injury caused by cerebral ischemia and reperfusion (FIG. 2).

1. A polypeptide having anti-thrombus activity, which is derived from Haemadipsa sylvestris antithrombotic peptide Sylvestin, comprising
(1) a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to the amino acid sequence set forth in SEQ ID NO: 1, or
(2) a sequence obtained by adding, deleting, or replacing one or more amino acids in the amino acid sequence set forth in SEQ ID NO: 1.

2. The polypeptide according to claim 1, comprising or consisting of the amino acid sequence set forth in SEQ ID NO: 1.

3. The polypeptide according to claim 1, comprising the amino acid sequence set forth in SEQ ID NO: 1, wherein the amino acid other than the amino acid set forth in SEQ ID NO: 1 is identical to the amino acid at the corresponding position of the amino acid sequence encoded by Haemadipsa sylvestris antithrombotic peptide cDNA in nature.

4. The polypeptide according to claim 1, which is produced by genetic engineering or chemical synthesis.

5.-7. (canceled)

8. A method for inhibiting FXIIa and/or kallikrein, comprising exposing the polypeptide according to claim 1 to FXIIa and/or kallikrein.

9. A method of preventing and/or treating a disease caused by blood coagulation, comprising administering the polypeptide according to claim 1 to a subject in need thereof.

10. The method according to claim 9, wherein the disease caused by blood coagulation is a disease caused by intravascular infarction and/or thrombus, for example, a disease selected from the group consisting of arteriosclerosis, atherosclerosis, coronary artery disease, stroke, myocardial infarction, cerebral infarction, cerebral ischemia and acute cerebral ischemia.


SEQUENCE LISTING (2 sequences)

SEQ ID NO: 1 - protein, 43 aa, Artificial
  The amino acid sequence of Sylvestin
TSEPVCACPKMLFWVCGKDGETYTHPCIAKCHNVEVEHDGKCK

SEQ ID NO: 2 - DNA, 316 bp, Artificial
  The DNA encoding the Haemadipsa sylvestris antithrombotic peptide
  misc_feature (55)..(183)
gggaacccgaaacgggcattcgagctcggtacccggggatcctctagagattaaacctcggaaccggtatgtgcatgcccaaaaatgctattttgggtttgtggtaaagatggtgagacttacacccatccttgcattgcaaaatgccataatgttgaagttgaacatgatgggaagtgcaaatgaaagggaccattcttcgaaattgcctgaaacttaaaaatattgatttgaatttaattaattcttattaattataacgtttcatcataataaatgaattacgaaaaaaaaaaaaaaaaaaaaaaaaaaaaaa